(19) United States
(12) Patent Application Publication
(10) Pub. No.: US 2017/0036001 A1
(43) Pub. Date: Feb. 9, 2017

(54) MATERNITY BELLY WRAP

(71) Applicant: Jodi Kocevar, Kirtland, OH (US)

(72) Inventor: Jodi Kocevar, Kirtland, OH (US)

(21) Appl. No.: 14/820,732

(22) Filed: Aug. 7, 2015

(51) Int. Cl. A61M 35/00; A41F 1/04; A41C 1/10

(52) U.S. Cl. CPC A61M 35/00; A41C 1/10; A41F 1/04

(57) ABSTRACT

A maternity belly wrap to wrap about the belly portion of a woman having moisturizer applied to skin of the belly portion. The maternity belly wrap includes a strip of material formed of at least one layer of material having a first end and second end. The strip of material has an outer surface and an inner surface and is formed of a material that prevents the moisturizer applied on skin of the belly potion from seeping through the material. The strip of material is elastic for maintaining a constant adherent traction with the belly portion of the woman and for preventing the skin of the belly portion from stretching apart and the elastic fibers of the skin from tearing. A closure system is included for securely fastening together the first and second ends of the strip of material.

### TECHNICAL FIELD OF THE INVENTION

The present invention relates to a maternity belly wrap. More specifically, the present invention relates to a belly wrap to wrap about the belly portion of a woman having moisturizer applied to skin of the belly portion.

### BACKGROUND OF THE INVENTION

Many women develop stretch marks on their abdomens, chests, legs, and other areas during pregnancy. Stretch marks are caused by the growth of the baby and rapid expansion of the skin around these areas. Factors that contribute to stretch marks include the age of the woman, the size of the baby, being pregnant with multiple babies and weight gained during pregnancy. Stretch marks are caused by tears or breakage of elastic fibers within the dermis. This commonly occurs as a chain reaction, wherein one elastic fiber rips causing nearby elastic fibers to rip. These multiple rips occur in a line, which is visible from the exterior of the skin as a stretch mark.

The quality of elastic fibers in a women's skin may be influenced by a women's age. For example, an eighteen year old girl will be more susceptible to stretch marks as she will have already stretched the fibers in her skin to near the breaking point. This is because she has just gone through growth spurts as a result of puberty and the skin has not had time to reinforce itself. However, slightly older women will have more time for the fibers to repair themselves after puberty and will therefore be less susceptible to stretch marks because their skin is reinforced. Many women have significantly more elastic fibers in their skin due to genetic factors. This is why some women get little or no stretch marks during pregnancy while others get severe stretch marks with the same size baby and same weight gain.

### SUMMARY OF THE INVENTION

According to an embodiment of the present invention, there is disclosed a maternity belly wrap to wrap about the belly portion of a woman having moisturizer applied to skin of the belly portion. The maternity belly wrap includes a strip of material formed of at least one layer of material having a first end and second end. The strip of material has an outer surface and an inner surface and is formed of a material that prevents the moisturizer applied on skin of the belly potion from seeping through the material. The strip of material is elastic for maintaining a constant adherent traction with the belly portion of the woman and for preventing the skin of the belly portion from stretching apart and the elastic fibers of the skin from tearing. A closure system is included for securely fastening together the first and second ends of the strip of material.

According to another embodiment of the present invention, there is disclosed a maternity belly wrap to wrap about the belly portion of a pregnant woman having moisturizer applied to skin of the belly portion. The maternity belly wrap includes a strip of material formed of a first inner layer and a second outer layer, the strip of material having a first end and second end. The inner layer of the strip of material is formed of a material that prevents the moisturizer applied on skin of the belly potion from seeping through the material. The first inner layer and the second outer layer are elastic for maintaining a constant adherent traction with the belly portion of the woman and for preventing the skin of the belly portion from stretching apart and the elastic fibers of the skin from tearing. A closure system is included for securely fastening together the first and second ends of the strip of material.

### BRIEF DESCRIPTION OF THE DRAWINGS

The structure, operation, and advantages of the present invention will become further apparent upon consideration of the following description taken in conjunction with the accompanying figures (FIGS.). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

FIG. 1 is a front view of the maternity belly wrap in use, in accordance with the present invention.

FIG. 2 is a rear view of the maternity belly wrap in use, in accordance with the present invention.

FIG. 3 is a rear three-dimensional view of a second embodiment of the maternity belly wrap, in accordance with the present invention.

FIG. 4 is a cross-sectional view through line 4-4 of FIG. 3, in accordance with the present invention.

### DETAILED DESCRIPTION OF THE PREFERRED EMBODIMENTS

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

In the drawings accompanying the description that follows, often both reference numerals and legends (labels, text descriptions) will be used to identify elements. If legends are provided, they are intended merely as an aid to the reader, and should not in any way be interpreted as limiting.

During pregnancy, a pregnant woman often suffers waist soreness and backache. It is caused by the change of the body attitude of the pregnant woman. In order to relieve the symptom of waist soreness and avoid low-lying fetal position and uterine prolapse, a pregnant woman often wears a maternity belt according to personal requirements. However, traditional maternity belts do nothing to prevent stretch marks, or the skin on the stomach from drying due to expansion. Currently, many pregnant women apply lotions and topical ointments to their skin as a preventative measure against stretch marks. However, lotions and topical ointments do not actually address the cause of stretch marks because they provide no reinforcement to prevent the skin's elastic fibers from ripping. Recently, research has been done on some specific skin care products, one being vitamin C, in the preventing and healing of stretch marks because it promotes collagen and healthy skin. The maternity belly wrap 10 will serve the purpose of keeping skin strengthening products on the skin and off of the users clothing helping prevent stretch marks.

The maternity belly wrap 10 is designed to wrap about the belly portion 12a (also called the abdomen and lower torso herein) of a pregnant woman 12. After a moisturizer or cream has been applied directly onto the skin of belly portion 12a, belly wrap 10 keeps moisture on the belly portion 12a for long periods of time so the moisturizer does not have to be reapplied throughout the day. Belly wrap 10 also helps prevent excessive itching due to dry skin and the skin being stretched so quickly. Further, the belly wrap 10 is designed for accommodating the changes in a woman's shape during various stages of pregnancy. The belly wrap 10 will prevent stretch marks from forming on the belly portion, abdomen and lower torso 12a of a woman 12.

The belly wrap 10 is a strip of material substantially tubular or cylindrical in shape and in a size so as to be expanded and stretched when worn by a pregnant woman 12. The belly wrap 10 is worn about the belly portion 12a of a woman 12 approximately at the waist. As illustrated in FIG. 2, the strip of material forming the belly wrap 10 has a first end 14 and second end 16 that are securely fastened together. However, it is within the terms of the embodiment that the strip of material forming the belly wrap 10 is constructed of one continuous length of material.

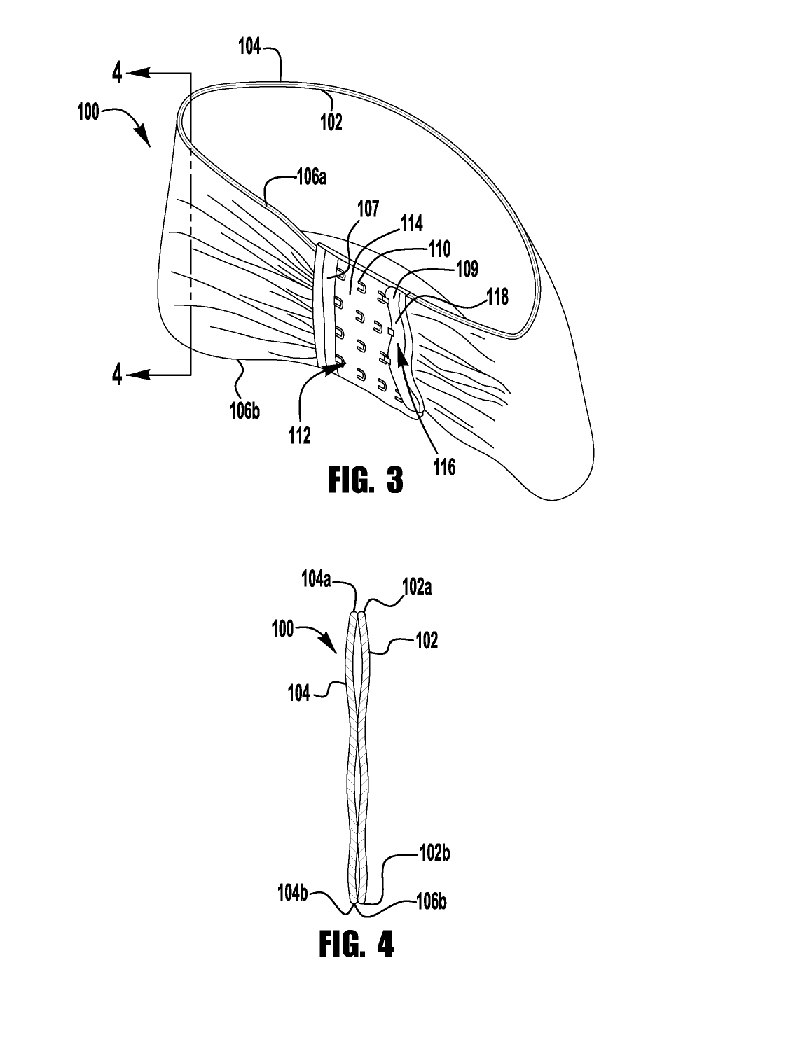

The belly wrap 10 is designed to be worn comfortably under a woman's 12 clothing. The strip of material forming the belly wrap 10 is constructed of at least one layer 18 of material or fabric having an outer surface 18a and an inner surface 18b. It should be noted that while only a single layer 18 of material is shown, it should be understood that the belly wrap 10 may consist of multiple layers of material as described with respect to FIGS. 3 and 4, wherein there are two layers of material as described. The inner surface 18b of the layer 18 of material is designed to maintain a constant adherent traction with the woman's belly portion 12a, to further assist in preventing the skin from stretching apart and the elastic fibers of the skin from tearing. The outer surface 18b of the layer 18 of material of belly wrap 18 is preferably smooth and non-adherent, thereby not adhering to outer clothing.

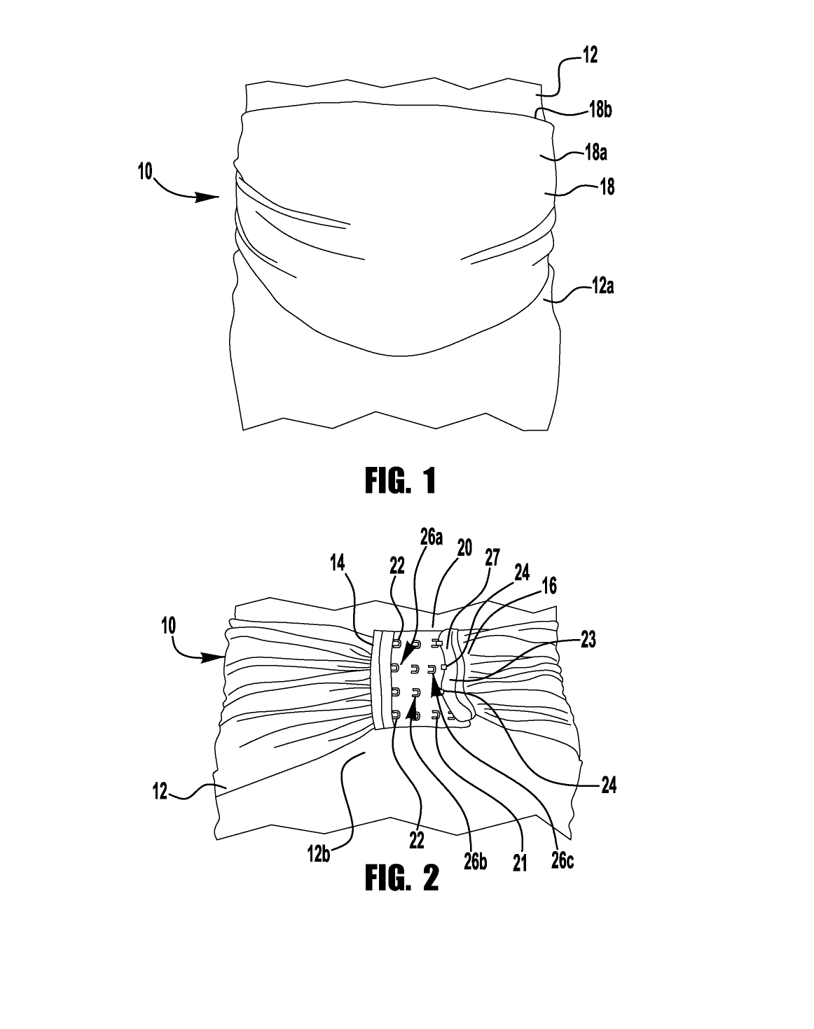

The strip of material 18 of the belly wrap 10 as shown in FIGS. 1 and 2 is constructed of stretchable material, preferably synthetic fiber materials selected from the group of materials including Lycra, spandex, nylon, polyester, and polyurethane laminate (PUL) or any other desired material that prevents the moisturizer, oil, or cream that is applied on the skin of the lower abdomen 12a from seeping through the material from the inner surface 18b to the outer surface 18b. While some of the stretchable materials may be breathable, some materials such as the flit material may not be breathable. The MIL material is a compound fabric made by laminating a cloth fabric to a thin film of polyurethane. Most PUL fabric is made by laminating lightweight polyester interlock knit fabric to a 1 mil thick film of polyurethane. There are two processes used for lamination are solvent lamination which fuses the fabric and polyurethane film into a single monolithic fabric and hot melt which uses heat activated glue to adhere the fabrics together. The elastic in the fabric of the belly wrap 10 comfortably hugs a woman's lower abdomen 12a. Typically, the material is designed to be firmly but comfortably secured about the woman's belly portion 12a so that it does not slip off of the belly portion 12a. Since the belly wrap 10 is constructed of stretchable material, it is designed such that one size fits most users. However, it is within the terms of the embodiment that two or three different sizes can be provided if desired, for different sizes of women.

The strip of material forming belly wrap 10 may have a uniform size, such as a width with a range between 12 inches and 40 inches, and a length with a range between 23 inches and 60 inches. Alternatively, the width of the strip of material forming belly wrap may be tapered with the widest part of the strip forming the belly wrap located at the front and center of the woman's belly 12a and the narrower part near the ends 14 and 16 disposed over center of the woman's back 12b, as illustrated in FIG. 2.

FIG. 1 illustrates a front view of the pregnant woman 12, at an advanced stage of pregnancy wearing the belly wrap 10. The belly wrap 10 works in conjunction with a moisturizer, oil, or cream that is applied on the skin of the lower abdomen 12a. Alternatively, the moisturizer may be applied to the inner surface 18b of the belly wrap 10, such that, when the belly wrap is worn around the belly portion 12a of a woman 12, the moisturizer is continuously applied. This generally provides users with application of significant amounts of oil and/or moisturizer to regions of the body that might suffer scarring or stretch marks due to pregnancy or weight changes.

The belly wrap 10 is designed to prevent the seepage of the oil, cream, and/or moisturizer to the woman's 12 outer clothing or garments. By wearing the belly wrap 10 under the woman's 12 outer clothing, the wearer may go about their daily lives while simultaneously enjoy the moisturizing effects of the cream. In this manner, the moisturizer is held against the woman's 12 skin and is not absorbed onto the outer clothing.

FIG. 2 illustrates a rear view of the pregnant woman 12, at an advanced stage of pregnancy wearing the belly wrap 10. While any desired means of interconnecting the ends of the belly wrap 10, in the embodiment illustrated in FIG. 2, a hook and eye closure system 20 is utilized to secure the belly wrap 10 after it had been wrapped around the belly portion 12a. Several series of loop elements 22 are affixed in a plurality of spaced vertical columns 26a, 26b and 26c to a strip 21 of material which is preferably stretchable and secured to the first end 14 of the belly wrap 10. A second series of hook elements 24 are arranged in a vertical column 27 and affixed to a strip 23 of material which can also be stretchable and secured to the second end 16 of the belly wrap 10. The spacing between the vertically disposed loop elements 22 and vertically disposed hook elements 24 are essentially the same so that the vertical column of hook elements 24 can be attached to any vertical column of loop elements. The hook elements 24 are commonly made of flattened wire bent to the required shape, and the loop elements 22 are also typically constructed of the same flattened wire bent into a shape into which the hooks can be attached. Because there is a plurality of columns 26a, 26b and 26c of loop elements 22 to which the column 27 of hook elements 24 can be secured, the belly wrap 10 is adjustable in size.

In an alternative embodiment, a hook and loop fastener system, such as Velcro, may be utilized to secure the belly wrap 10 after it had been wrapped around the belly portion 12a. A loop portion (not shown) is permanently attached to a first strip of stretchable fabric which in turn is secured to the first end 14 of the belly wrap 10, and a hook portion (not shown) is permanently attached to an underside of a second strip of fabric which in turn is attached to the second end 16 of the belly wrap. When the loop portion and the hook portion are pressed together, the hooks catch in the loops and the two pieces fasten or bind temporarily during the time that they are pressed together. This hook and loop fastener system allows the belly wrap 10 to be adjustable to accommodate a variety of body sizes.

However, it is also within the terms of the embodiment that the belly wrap 10 be one continuous elasticized length of material that the woman would simply fit about her body. Alternatively, the belly wrap 10 can utilize a buckle arrangement, or any other fastener effective to hold the wrap around the torso of the user.

FIGS. 3 and 4 illustrate a second embodiment of the maternity belly wrap 100. In this embodiment, the belly wrap 100 is constructed of a strip of material formed of first and second layers, 102 and 104 respectively, with a first and second end 107 and 109, respectively. The first inner layer 102 is designed to designed to maintain a constant adherent traction with the woman's belly portion, and to further assist in preventing the skin from stretching apart and the elastic fibers of the skin from tearing. Further, a moisturizer is applied to the first layer 102 such that, when the belly wrap 100 is worn around the belly portion 12a of a woman 12, the moisturizer is continuously applied. The first layer 102 prevents the moisturizer or cream that has been applied to the woman's lower abdomen 12a from seeping therethrough, drying out and also tarnishing the woman's clothes.

Typically, herein the first layer 102 is constructed of stretchable breathable synthetic fiber materials selected from a group of materials including Lycra, spandex, nylon, polyester and polyurethane laminate (PUL). The second or outer layer 104 can be constructed of any stretchable material. If desired, it too can be constructed of a stretchable breathable synthetic fiber materials selected from a group of materials including Lycra, spandex, nylon, polyester and polyurethane laminate (PUL) as with the first or inner layer 102. It is within the terms of the strip of material formed with first and second layers, 102 and 104, respectively, be of a uniform size. Alternatively, the width of the strip of material forming the belly wrap may be tapered with a widest part of the strip designed to be disposed at a front and center of the woman's belly portion 12a and a narrower part of the strip near the first and second ends 107 and 109 disposed near the center of a woman's back.

Typically, the first inner layer 102 and second outer layer 104 of the strip are joined along the first and second edges 102a and 102b of inner layer 102 to first and second edges 104a and 104b of outer layer 104, respectively, in any desired fashion, preferably with sewing or an adhesive. However, in all other manners, the second embodiment of the maternity belly wrap 100 is identical to the first embodiment of the maternity belly wrap 10, such as means for securely fastening the first end and second ends 107 and 109 of the first and second layers, 102 and 104, respectively.

Referring again to FIG. 3, a hook and eye closure system 110 is utilized to secure the belly wrap 100 after it had been wrapped around the belly portion 12a. Several series of loop elements 112 are affixed in a plurality of spaced vertical columns to a strip 114 (compare FIG. 1) of material which is preferably stretchable and secured to the first end 107 of the belly wrap 100. A second series of hook elements 116 are arranged in a vertical column and affixed to a strip 118 of material which can also be stretchable and secured to the second end 109 of the belly wrap 100. The spacing between the vertically disposed loop elements 112 and vertically disposed hook elements 24 are essentially the same so that the vertical column of hook elements 116 can be attached to any vertical column of loop elements 114. The hook elements 114 are commonly made of flattened wire bent to the required shape, and the loop elements 116 are also typically constructed of the same flattened wire bent into a shape into which the hooks can be attached. Because there is a plurality of columns of loop elements 114 to which the column of hook elements 116 can be connected, the belly wrap 10 is adjustable in size.

In use, the woman 12 applies any desired moisturizer or cream to her belly portion 12a. Then, she fastens the first and second ends 14 and 16, of the maternity belly wrap 10, respectively, around her belly portion 12a. The maternity belly wrap 10 is secured around the pregnant woman's belly portion 12a with the hook and eye closure system 20 or a hook and loop fastener system. The moisturizer is then contained and continuously applied to the pregnant woman within the maternity belly wrap 10.

In use of the second embodiment, the woman 12 applies any desired moisturizer or cream to her belly portion 12a or the inner layer of material 102. Then, she fastens the first and second ends 107 and 109, of the maternity belly wrap 100, respectively, around her belly portion 12a The maternity belly wrap 100 is secured around the pregnant woman's belly portion 12a with the hook and eye closure system 110 or a hook and loop fastener system (not shown). The moisturizer is then contained and continuously applied to the pregnant woman within the maternity belly wrap 100.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

What is claimed is:

1. A maternity belly wrap to wrap about the belly portion of a woman having moisturizer applied to skin of the belly portion, comprising;
a strip of material formed of at least one layer of material having a first end and second end;
the strip of material having an outer surface and an inner surface and being formed of a material that prevents the moisturizer applied on skin of the belly potion from seeping through the material;
the strip of material being elastic for maintaining a constant adherent traction with the belly portion of the woman and for preventing the skin of the belly portion from stretching apart and the elastic fibers of the skin from tearing; and
a closure system for securely fastening together the first and second ends of the strip of material.

2. The maternity belly wrap of claim 1 wherein the strip of material forming the belly wrap is substantially tubular or cylindrical in shape and in a size so as to be expanded and stretched when worn around a pregnant woman.

3. The maternity belly wrap of claim 2 wherein the least one layer is constructed of a stretchable, breathable synthetic fiber material selected from a group of materials comprising Lycra, spandex, nylon, polyurethane laminate and polyester.

4. The maternity belly wrap of claim 2 wherein the strip of material is of a uniform size.

5. The maternity belly wrap of claim 2 wherein a width of the strip of material forming the belly wrap belly is tapered with a widest part adapted to being located at a front and center of the woman's belly portion and a narrower part toward the first and second ends of the strip adapted to being located near the center of the woman's back.

6. The maternity belly wrap of claim 1 wherein the moisturizer is applied to the inner surface of the strip of material such that, when the belly wrap is worn around the belly portion of a woman, the moisturizer is continuously applied to the skin of the belly portion.

7. The maternity belly wrap of claim 1 wherein the closure system is a hook and eye closure system utilized to secure the belly wrap after it had been wrapped around the belly portion of a pregnant woman.

8. The maternity belly wrap of claim 7 wherein the hook and eye closure system includes:
a series of loop elements affixed in a plurality of spaced vertical columns to a first stretchable strip of material secured to the first end of the belly wrap; and
a series of hook elements arranged in a vertical column and affixed to a second strip of stretchable material and secured to the second end of the belly wrap.

9. The maternity belly wrap of claim 8 wherein spacing between the series of loop elements in the vertical columns and spacing between the series of hook elements disposed in a vertical column are essentially the same so that the vertical column of hook elements can be attached to any of the vertical column of loop elements.

10. The maternity belly wrap of claim 9 wherein the hook elements are made of flattened wire bent to the required shape, and the loop elements are made of the same flattened wire bent into a shape into which the hooks can be attached.

11. A maternity belly wrap to wrap about the belly portion of a pregnant woman having moisturizer applied to skin of the belly portion, comprising;
a strip of material formed of a first inner layer and a second outer layer;
the strip of material having a first end and second end;
the inner layer of the strip of material being formed of a material that prevents the moisturizer applied on skin of the belly potion from seeping through the material;
the first inner layer and the second outer layer being elastic for maintaining a constant adherent traction with the belly portion of the woman and for preventing the skin of the belly portion from stretching apart and the elastic fibers of the skin from tearing; and
a closure system for securely fastening together the first and second ends of the strip of material.

12. The maternity belly wrap of claim 11 wherein the first inner layer and the second outer layer are joined along first and second edges of the first inner layer and the second outer layer, respectively.

13. The maternity belly wrap of claim 11 wherein the belly wrap is substantially tubular or cylindrical in shape and in a size so as to be expanded and stretched when worn around a pregnant woman.

14. The maternity belly wrap of claim 13 wherein the first inner layer and second outer layer are constructed of stretchable breathable synthetic fiber materials selected from a group of materials comprising Lycra, spandex, nylon, polyurethane laminate and polyester.

14. (canceled)

15. The maternity belly wrap of claim 11 wherein a width of the strip of material forming the belly wrap belly is tapered with a widest part adapted to being located at a front and center of the woman's belly portion and a narrower part toward the first and second ends of the strip adapted to being located near the center of the woman's back.

16. The maternity belly wrap of claim 11 wherein the moisturizer is applied to the inner surface of the first layer of the strip of material such that, when the belly wrap is worn around the belly portion of a woman, the moisturizer is continuously applied to the skin of the belly portion.

17. The maternity belly wrap of claim 11 wherein a hook and eye closure system is utilized to secure the belly wrap after it had been wrapped around the belly portion of the pregnant woman.

18. The maternity belly wrap of claim 17 wherein the hook and eye closure system includes:
a series of loop elements affixed in a plurality of spaced vertical columns to a first stretchable strip of material secured to the first end of the belly wrap; and
a series of hook elements arranged in a vertical column and affixed to a second strip of stretchable material and secured to the second end of the belly wrap.

19. The maternity belly wrap of claim 18 wherein spacing between the series of loop elements in the vertical columns and spacing between the series of hook elements disposed in a vertical column are essentially the same so that the vertical column of hook elements can be attached to any of the vertical column of loop elements.

20. The maternity belly wrap of claim 19 wherein the hook elements are made of flattened wire bent to the required shape, and the loop elements are also typically constructed of the same flattened wire bent into a shape into which the hooks can be attached.
